(19) United States
(12) Patent Application Publication
(10) Pub. No.: US 2024/0139475 A1
(43) Pub. Date: May 2, 2024

(54) DEVICE, METHOD, AND SYSTEM FOR SECURING A CATHETER COVER

(71) Applicant: Cath Dry, Inc., Los Angeles, CA (US)

(72) Inventor: Pascal Dabel, Los Angeles, CA (US)

(21) Appl. No.: 18/050,123

(22) Filed: Oct. 27, 2022

(51) Int. Cl. A61M 25/02

(52) U.S. Cl. CPC A61M 25/02; A61M 2025/0246; A61M 2025/0266; A61M 2025/028

(57) ABSTRACT

A sterile, water-resistant, universal, hypoallergenic, soft and flexible device that embodies a method and system for securing a catheter dressing to a patient's body to prevent the catheter dressing from being unintentionally removed and dislodge, whereby the securement device provides a second connection point to the patient. The device embodies a cushioned impression that receives a sheathed catheter hub. A top flap of the devices moves to a closed position, whereby only the sheath is directly engaged by the top flap.

### BACKGROUND OF THE INVENTION

The present invention relates to catheter dressings and, more particularly, to a sterile, water-resistant, universal, hypoallergenic, soft and flexible device that embodies a method and system for securing a catheter dressing to a patient's body, preventing unintentional removal or dislodgment of the catheter dressing.

A peripherally inserted central catheter (PICC) is a thin, flexible tube that is inserted into a central vein of a medical patient. It is used to give intravenous fluids, blood transfusions, chemotherapy, drugs and to draw blood samples. Such patients that must use a PICC or other catheters are not allowed to safely shower or swim for weeks or months at a time. The patients thereby become dirty, uncomfortable, and are more prone to infection. The only way a patient may clean themselves is by using a washcloth or sponge which promotes cross contamination of the catheter site. Further, the patients are at increased risk for blood stream infections since there are no appropriate methods of protecting the catheter site.

Thanks in part to the inventor's U.S. Pat. No. 10,137,292 and related work, methods of covering the catheter now include a water-resistant catheter dressing that the catheter slides into for protection against the elements, wherein a proximal end of the catheter dressing is adhered to the patient. However, the catheter dressings themselves can be a source of failure or exposure of the catheter site to infection is inadvertently removed or disclosed from the patient. Specifically, the catheter dressing is typically a water-resistant sleeve, wherein along the rim of the sleeve an adhesive structure is disposed to connect the catheter dressing to the patient. Inadvertent force applied to the sleeve can translate to the adhesive structure be pulled away from the patient or otherwise compromised.

Current securement solutions for catheters are devices that are commonly applied directly to or around the PICC or catheter hub, then the dressing must be applied over these devices. These solutions sometimes prevent the use of a catheter dressing and have the disadvantage of being catheter-specific as opposed to being catheter-neutral or universal.

Furthermore, it is known in the art relating to catheters that after insertion of a catheter through a patient's skin into a vein such as a central vein (CVC), the catheter is ideally secured to prevent the catheter from slipping out of the patient's vein, which can happen when the catheter and its hub feels tension or pulling forces when the patient is mobile. Commonly, a catheter securement device is used to hold the catheter hub to the patient's skin. However, when the catheter hub is already sheathed in the sterile dressing of the present invention, conventional catheter securement devices would damage or disturb the sterile dressing, frustrating the sterile dressing's purpose.

As can be seen, there is a need for a universal, sterile, water-resistant, hypoallergenic, soft, and flexible device that embodies a method and system for securing a catheter dressing to a patient's body to prevent the catheter dressing from being unintentionally removed or having its adhesive structure connecting the catheter dressing to the patient compromised.

### SUMMARY OF THE INVENTION

One object of the present invention is a catheter dressing securement device that securely anchors a catheter dressing to the patient's skin wherein the securement device is applied around and externally to the catheter dressing, thereby providing a second point of connection to the patient, and as a result, the catheter dressing securement device may be universal in that it does not directly contact the catheter already covered by the catheter dressing.

Another object of the present invention is that the catheter dressing securement device is made from material that is sterile, water-resistant, hypoallergenic, soft, and flexible.

An object of the present invention also includes that the catheter dressing securement device has a low profile through obviating the need and so not having clips or hard components that could damage the catheter dressing.

Likewise, another object of the present invention is an interface portion of the securement device that engages the external surface of the catheter dressing, and wherein this interface portion has no adhesive element, so the dressing does not get stuck or damaged.

One object of the present invention is to have two paper liners along a rear adhesive surface of a base of the securement device for peeling away the two paper liners to adhere the securement device directly to the patient.

Another object of the present invention is to have a cushioned impression formed in an anchoring body of the securement device to protectively nest the pre-covered catheter hub within the cushioned impression, thereby locking the pre-covered catheter in place against both accidental vertical and horizontal lifting and pulling as well as other lateral forces.

Yet another object of the present invention is to provide a top flap to fold over the pre-covered catheter hub, encasing it with the cushioned impression, wherein the supporting surface of the cushioned impression operatively associates with a connector of the top flap, making the top flap self-gripping, and so providing further resistance to accidental vertical and horizontal lifting and pulling.

The present catheter securement device is designed to be used to anchor catheter hubs sheathed in a water-resistant catheter dressing. For example, one intended use of the present catheter dressing securement device is to secure PICCs sheathed in the water-resistant sleeve. The present catheter dressing securement device prevents stresses applied to the catheter hub or fittings thereof and thus stops catheter migration or removal as well as insertion site irritation, by way of providing a second connection to the patient, and thereby modifying the load path of unintentional forces acting on the catheter dressing so these unintentional forces are carried by the securement device of the present invention.

In one aspect of the present invention, a device for securing a catheter sheathed in a catheter dressing, the device includes the following an anchoring body configured to be mounted on a patient; two opposing wing portions connected to the anchoring body; a cushioned impression defined by the anchoring body, wherein the impression is configured to receive a hub of the catheter sheathed in the catheter dressing; and a top flap connected to one of said wing portions so as to be movable from an open position to a closed position overlapping the cushioned impression.

In another aspect of the present invention, the device for securing a catheter sheathed in a catheter dressing, further includes wherein the closed position urges a portion of the catheter dressing against the top surface, wherein the top flap is self-gripping, wherein a first connector is provided along the top flap, wherein the first connector is attachable to the opposing wing portion and an exterior of the catheter dressing; and further including a second connector disposed along the opposing wing portion, wherein a shape the cushioned impression is defined by a top surface of the anchoring body, and wherein an elevation of the cushioned impression is defined by a distance from the top surface and a bottom surface, and wherein the elevation is designed to be a distance equal or greater than a thickness of the catheter hub, and wherein the shape can have two or more arcuate portions.

In yet another aspect of the present invention, a method of securing a catheter to a patient, wherein the catheter is sheathed in a catheter dressing, the method includes providing the above-mentioned device; securing a catheter hub of the catheter in the cushioned impression said device; moving the top flap to the closed position, encasing the catheter hub in the cushioned impression; and mounting the device to the patient, wherein the mounted device forms a second patient connection point spaced apart form a first connection patient point initially established by the catheter dressing.

These and other features, aspects and advantages of the present invention will become better understood with reference to the following drawings, description and claims.

### BRIEF DESCRIPTION OF THE DRAWINGS

FIG. 1 is a is a front elevation view of an exemplary embodiment of the catheter dressing securement device of the present invention.

FIG. 2 is a detailed front elevation view of an exemplary embodiment of a retaining strap of the catheter dressing securement device of the present invention.

FIG. 3 shows two front elevation views of exemplary embodiments of the catheter dressing securement device of the present invention, illustrating various shapes of the cushioned impression 44 are contemplated herein.

FIG. 4 is a rear elevation view of an exemplary embodiment of the catheter dressing securement device of the present invention, illustrating peel-away liners along the adhesive rear surface.

FIG. 5 is a front elevation view of an exemplary embodiment of the catheter dressing securement device of the present invention, shown in use in a closed position.

FIG. 6 is a front elevation view of an exemplary embodiment of the catheter dressing securement device of the present invention, shown in use in another closed position, illustrating the adjustability of the retaining strap 50 (in view of FIG. 4) through a user selectively engaging the first connector of the retaining strap 50 relative to the second wing portion.

FIG. 7 is a front elevation view of an exemplary embodiment of the catheter dressing securement device of the present invention.

FIG. 8 is a detailed front elevation view of an exemplary embodiment of the cushioned impression 44 of the catheter dressing securement device of the present invention.

FIG. 9 shows a series of diagrammatical view elevation views of an exemplary embodiment, visually illustrating the process of applying the catheter dressing securement device of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims as may be interpreted in light of the description and the common general knowledge in the relevant technical field such as medicine.

Broadly, an embodiment of the present invention provides a sterile, water-resistant, universal, hypoallergenic, soft, and flexible device that embodies a method and system for securing a catheter dressing to a patient's body, whereby the securement device provides a second connection point to the patient, to prevent the catheter dressing from being unintentionally removed and dislodge. The device embodies a cushioned impression that receives a sheathed catheter hub. A top flap of the devices moves to a closed position, whereby only the sheath is directly engaged by the top flap.

More particularly, a catheter dressing securement device in accordance with the present invention includes a flexible base member having an adhesive side and an opposite non-adhesive side. The catheter dressing securement device has an anchoring body made from cushioned material with a generally flat top surface. The anchoring body defines a cushioned impression or recess for receiving the catheter hub that is already covered by a catheter dressing. A wing portion may be provided on both sides of the anchoring body. A retention strap may be pivotably connected to one wing portion so as to be movable between an open position and a closed position secured to the other wing portion, and thereby overlapping the cushioned impression. The retaining strap provides a top connector that securely fastens to the anchoring body and the opposing wing portion, wherein the top connector attaches to the material of the anchoring body including a bottom surface of the cushioned impression.

The present invention may include a securement device 100 for a catheter dressing device 10. The catheter dressing device 10 may include a water-resistant sleeve 16. The water-resistant sleeve 16 may include a first end and a second end. The first end may include a rim forming an opening into the sleeve 16. The second end may be a removably closed end. The water-resistant sleeve 16 may be made of a polymer, such as a plastic. For example, the water-resistant sleeve 16 may be made of an Ultraviolet protective polyethylene. In certain embodiments, the water-resistant sleeve 16 may be approximately five to twelve inches long with about a one-inch diameter. The sleeve 16 may be formed to fit over the catheter 22 of a user 20. An adhesive structure 12 is attached to the rim of the water-resistant sleeve 16. The adhesive structure 12 attaches to the skin of the user 20 that surrounds the catheter 22. In certain embodiments, the adhesive structure 12 is a waterproof tape, adhesive film, or the like.

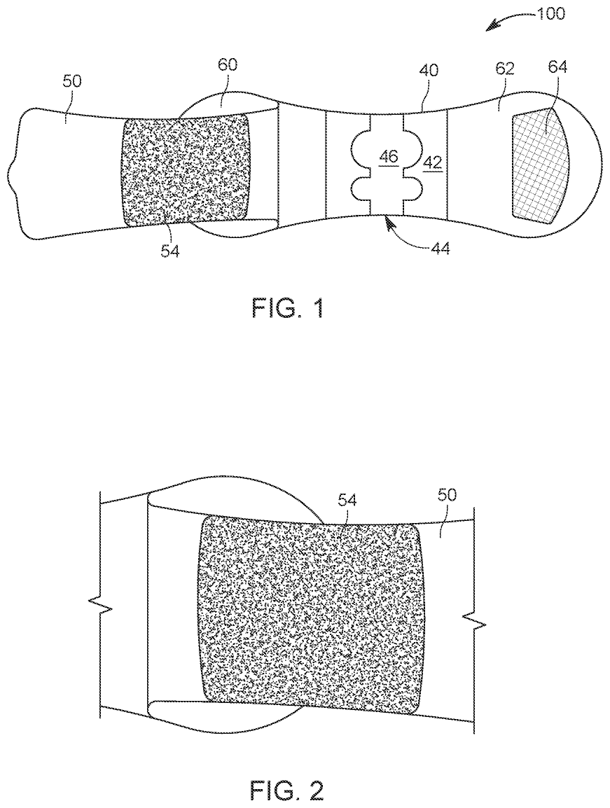

Referring to FIGS. 1 through 9, the present invention may include a catheter cover securement device 100 in accordance with the present invention. The securement device 100 may have an anchoring body 40 having rear side and a front side providing a cushioned impression 44. The rear side may have adhesive for removably attaching to the skin of a user 20. The rear side may have one or more peel-away tabs 61 that cover the adhesive prior to deployment, as illustrated in FIG. 4, for maintaining the adhesive properties of the adhesive until use.

The cushioned impression 44 may be defined by the build-up of cushioned material that terminates along a flat top surface 42. The cushioned impression 44 is dimensioned to receive a catheter hub (not shown) so that a received catheter hub is at an elevation equal to or less than the elevation of the flat top surface 42, relative to a bottom surface 46 of the cushioned impression 44 (that defines is lowest elevation).

One side of the anchoring body 40 may have a first wing portion 60 and the other side an opposing wing portion 62. The wing portions 60 and 62 may have adhesive on their rear surfaces. The second wing portion 62 may provide a second connector 64. The retaining strap 50 provides a first connector 54. The retaining strap 50 is movable between an open position and a closed position wherein the first connector 54 covers the cushioned impression 44 and extends past the cushioned impression 44 to removably attached to the second connector 64 and/or the second wing portion 62. In the closed condition, a catheter hub recessed in the cushioned impression 44 would be encased, closing off the opening through which the catheter hub was received. In certain embodiments, the top surface 42 may be domed, convex, or the like.

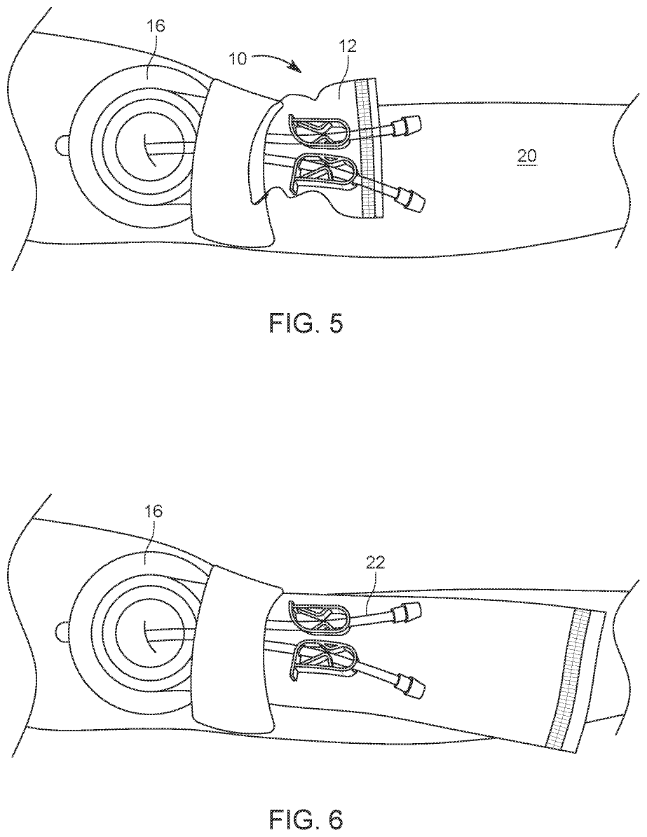

The rear side of the securement device 100 may be adhered to the skin of the user 20 spaced apart from the adhesive structure 16 to receive the catheter hub in the cushioned impression 44. The catheter hub of a catheter 20 sheathed in the cover sleeve 12 may be received in the cushioned impression 44 so that the retaining strap 50 closes around the exterior surface of the sleeve, as shown in FIGS. 4 and 5. This sandwiching of a portion of the catheter cover 10 helps stabilize it from various horizontal, vertical, or combination forces that may inadvertently pull on the sleeve 12 of the catheter cover 10. With the attachment of the adhesive structure 16 and the securement device 100, two connections points are made to the user 20, thereby resisting rotational (moment) forces, lateral forces, etc.

The retaining strap 50 can be woven cotton, rayon fabric, woven material. The first connector 54 may be adapted to not only removable connect to the second connector 64, but also connect to the anchoring body 40, including the bottom surface 46, making the retaining strap 50 self-gripping. Once the catheter hub is secured by the securement device 30, stresses and forces applied in any direction on the fittings connected to the catheter 22 will not cause the catheter 22 to move and thereby irritate the insertion site.

The first connector 54 may define an engagement surface that is dimensioned and adapted to be generally or approximately coextensive with the top surface 42 as well as has additional length to extend to the second connector 64. Accordingly, as a result of the retaining strap 50 being moved to the closed position, the engagement surface interfaces with the top surface, closing off access to the cushioned impression 44. In certain embodiments, the top surface 42 may be domed, convex, or the like, and the engagement surface 52 of the retaining strap 50 adapted to conform to the domed top surface 42.

The anchoring body 40 may be made of any suitable flexible, stretchable elastomeric material, or the like. The anchoring body 40 may be mounted on a non-adhesive side of a base member. The present invention may include a flexible base member having an adhesive side for adherence to a surface such as a patient's skin, and an opposite non-adhesive side. The surface of the adhesive side includes an adhesive such as a medical grade adhesive or similar thereon. The base member may be a generally flat, planar pad made of a foam, film, or the like. The wing portions 60 and 62 may be opposing extensions of the base member.

The retaining strap 50 may have a flat engagement surface coextensive with the first connector 52, which may be a coating of adhesive. Further, the flat engagement surfaces each have a longitudinal length equal to or greater than a width of the sleeve 16 so that in the closed condition the first connector 52 engages a substantial portion of said width.

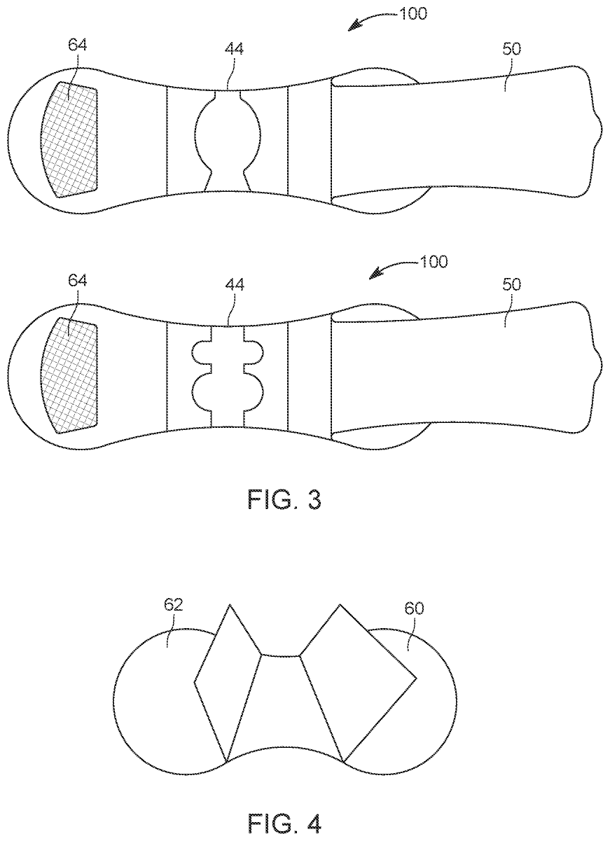

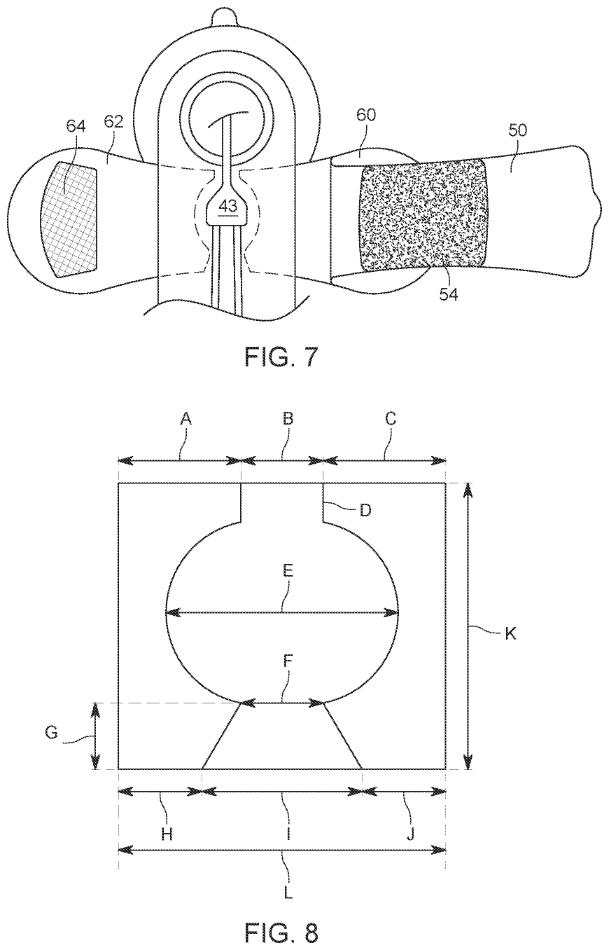

The cushioned impression 44 may be centrally disposed relative to the top surface 42. The anchoring body 40 may be generally symmetrical about a longitudinal axis and/or a transverse axis (i.e., x and y axes) of the securement device 100. Referring to FIG. 7, the shape and dimensions of the cushioned impression 44 may be A=approximately 1.25 cm; B=approximately 1.00 cm; C=approximately 1.25 cm; D=approximately 0.33 cm; E=approximately 2.00 cm; F=approximately 1.00 cm; G=approximately 0.67 cm; H=approximately 1.00 cm; I=approximately 1.50 cm; J=approximately 1.00 cm; K=approximately 3.00 cm; and L=approximately 3.50 cm. Again, it is understood that other shapes may be employed by the cushioned impression, as illustrated in FIGS. 3, as long as the can receive the catheter hub.

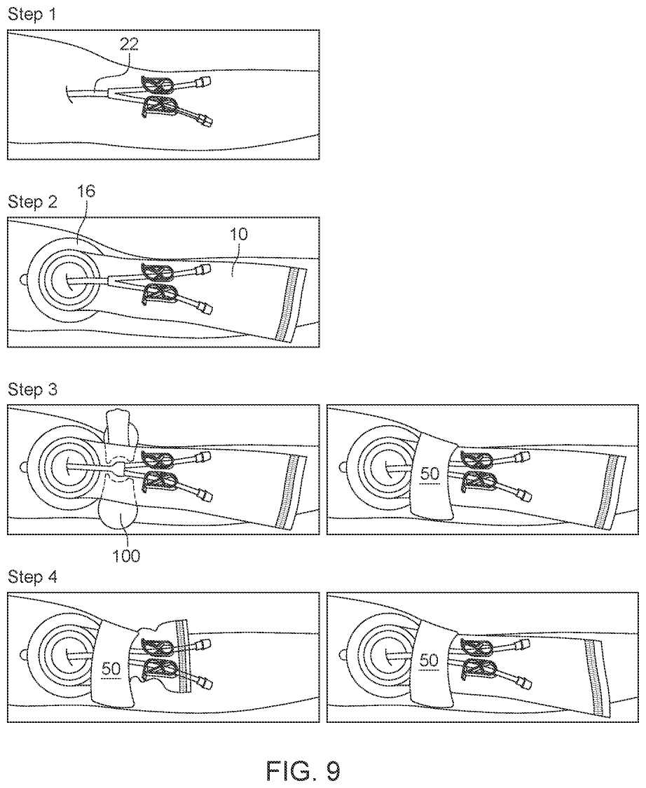

Referring to FIG. 9, a method of using the catheter cover securement device 100 may include the following. First a user may be provided the catheter covering securement device 100 disclosed above. After the patient has received the catheter 22 (Step 1 in FIG. 9) that has then been received in a catheter cover 10 (Step 2 in FIG. 9), the user may groom an area of the body downward of the adhesive structure 16 of the catheter cover 10. Per step 3 in FIG. 9, the user may adhere the securement device 100 by way of its adhesive rear side/surface to the skin of the user so that the cushioned impression 44 aligns with the catheter hub 43, wherein the securement device 100 is underneath the catheter cover 10, as illustrated in the first panel of Step 3 of FIG. 9). The user may urge the catheter hub and the associated portion of the sleeve 12 of the catheter cover 10 into the cushioned impression 44, as illustrated in the second panel of Step 3 of FIG. 9).

As shown in Step 4 of FIG. 9, then the user may move the retaining strap 50 to the closed position so that the first connector attaches to the opposing, second wing 62. The closed position secures the catheter hub housed in the cushioned recess 44 and the exterior of the cover sleeve engaged by the first connector 54 and presses against the top surface 52 of the securement device 100 so that an effect second connection point on the skin of the patient is established. The retaining strap 50 may be selectively pulled tight (as reflected in the first panel of Step 4 of FIG. 9) or more loosely (as reflected in the second panel of Step 4 of FIG. 9) depending on the patient's comfort level. Further securing the catheter to the patient so that inadvertent pulls do not dislodge the catheter cover's connection to the patient 20 (at the adhesive structure 16). Since the first connector 54 is adapted to adhere to the top and bottom surfaces 52 and 46 of the anchoring body 40 and the second wing portion 62 (as well as the exterior of the cove) of the present invention is considered self-gripping. Note, a second connector 64 is not required to facilitate the secured, closed position.

As used in this application, the term “about” or “approximately” refers to a range of values within plus or minus 10% of the specified number. And the term “substantially” refers to up to 90% or more of an entirety. Recitation of ranges of values herein are not intended to be limiting, referring instead individually to any and all values falling within the range, unless otherwise indicated, and each separate value within such a range is incorporated into the specification as if it were individually recited herein. The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

As used in the specification and the claims, the singular form “a,” “an” and “the” include singular and plural references unless the context clearly dictates otherwise.

It should be understood, of course, that the foregoing relates to exemplary embodiments of the invention and that modifications may be made without departing from the spirit and scope of the invention as set forth in the following claims.

What is claimed is:

1. A device for securing a catheter sheathed in a catheter dressing, the device comprising:
an anchoring body configured to be mounted on a patient;
two opposing wing portions connected to the anchoring body;
a cushioned impression defined by the anchoring body, wherein the impression is configured to receive a hub of the catheter sheathed in the catheter dressing; and
a top flap connected to one of said wing portions so as to be movable from an open position to a closed position overlapping the cushioned impression.

2. The device of claim 1, wherein the closed position urges a portion of the catheter dressing against the top surface.

3. The device of claim 2, wherein the top flap is self-gripping.

4. The device of claim 3, wherein a first connector is provided along the top flap, wherein the first connector is attachable to the opposing wing portion and an exterior of the catheter dressing.

5. The device of claim 4, further comprising a second connector disposed along the opposing wing portion.

6. The device of claim 2, wherein a shape the cushioned impression is defined by a top surface of the anchoring body, and wherein an elevation of the cushioned impression is defined by a distance from the top surface and a bottom surface, and wherein the elevation is designed to be a distance equal or greater than a thickness of the catheter hub.

7. The device of claim 6, wherein the shape can have two or more arcuate portions.

8. A method of securing a catheter to a patient, wherein the catheter is sheathed in a catheter dressing, the method comprising:
providing the device of claim 1;
securing a catheter hub of the catheter in the cushioned impression said device;
moving the top flap to the closed position, encasing the catheter hub in the cushioned impression; and
mounting the device to the patient.

9. The method of claim 8, wherein the mounted device forms a second patient connection point spaced apart form a first connection patient point initially established by the catheter dressing.
